(19) United States
(12) Patent Application Publication
(10) Pub. No.: US 2025/0049074 A1
(43) Pub. Date: Feb. 13, 2025

(54) EDIBLE MICROCARRIER FOR THE PREPARATION OF CULTURED MEAT AND METHOD OF PRODUCING SAME

(71) Applicant: Mirai International AG, Zürich (CH)

(72) Inventors: Alexander MOREL, Olten (CH); Janick Simon EGLAUF, Baden (CH); Nicolò RAZZA, Au ZH (CH); Suman Kumar DAS, Thalwil (CH)

(73) Assignee: Mirai International AG, Zürich (CH)

(21) Appl. No.: 18/717,733

(22) PCT Filed: Dec. 6, 2022
(86) PCT No.: PCT/EP2022/084525

§ 371 Date: Jun. 7, 2024

(30) Foreign Application Priority Data
Dec. 10, 2021 (EP) 21213775.6

(51) Int. Cl. A23J 3/22; A23L 13/00; C12N 5/077

(52) U.S. Cl. CPC A23J 3/227; A23L 13/00; C12N 5/0653; C12N 5/0659; C12N 2513/00; C12N 2531/00; C12N 2533/74; C12N 2533/76; C12N 2537/10

(57) ABSTRACT

Edible microcarrier for growing anchorage-dependent cells to prepare a cultured meat product, where the edible microcarrier includes a microcarrier core and a microcarrier coating at least partially directly or indirectly covering the surface of said microcarrier core, wherein said microcarrier core is a hydrogel having only the following components: (A) 0.03-10.0 wt % of at least one ionically crosslinked polysaccharide; (B) 0.05-5 wt % of a mechanical stability agent different from (A); (C) 0.0-10 wt % of additives different from (A) and (B); (D) 0.0-50 wt % living cells; (E) 75-99.92 wt % of water; where the sum of (A)-(E) making up 100 wt %, and where the microcarrier coating includes a biopolymer, where the biopolymer is crosslinked using a crosslinking agent.

### TECHNICAL FIELD

The present invention relates to edible microcarriers for growing anchorage-dependent cells to prepare a cultured meat product, to methods of producing such microcarriers, to uses of such microcarriers and to cultured meat products being based on such microcarriers. Following, the present disclosure relates generally to microcarriers for culturing cells and more specifically, it relates to edible microcarriers to efficiently cultivate cells at high density in bioreactors, methods of producing the microcarriers and using them to produce engineered meat products.

### PRIOR ART

Meat is an important protein source of the human diet. Due to controversial animal welfare associated with the traditional meat industry along with the increasing global population and demand for meat products, sustainable production alternatives are indispensable. Tissue engineering technology provides an opportunity to produce edible sources of animal protein that are not associated with the environmental degradation of livestock. However, culturing meat from cell culture faces important cell culture challenges as well as scale-up limitations. Microcarriers are the most promising candidates for upscaling cultivation of anchorage-dependent cells in bioreactors as microcarriers offer a large surface/volume ratio providing anchorage or an attachment surface for the suspended cell culture in a bioreactor. Microcarriers may be in the form of beads and can be manufactured from plant-based materials (e.g., cellulose), animal-based materials (e.g., gelatin), or synthetic materials (e.g., plastic, glass). Currently available microcarriers have been essentially developed to produce pharmaceutically active substances from cells cultured in a synthetic medium. In these applications, microcarriers support the growth of the cells that continuously produce the targeted molecules, while the targeted molecule is extracted and purified as the final product. However, as cells are part of the final product in cultured meat applications, microcarriers are used differently than in pharmaceutical production. For the preparation of cultured meat products, (i) microcarriers may serve as a temporary substrate for the cell growth and need then to be separated and filtered out from the cells at a particular stage of the culturing process; (ii) microcarriers may serve as a temporary substrate for the cell growth and are then degraded or dissolved at a particular stage of the culturing process to release the cells; (iii) microcarriers may remain to be embedded in the final product. Microcarriers disclosed in the prior art are not appropriate for large-scale production of cultured meat, as the microcarriers do not provide cost-effective, food compatible and process-efficient solutions. Existing microcarriers are not produced using only materials and processes that are fully approved for food consumption and thus require an extensive filtration process after the cell growth to avoid them or part of them ending up in the final product. Even the preparation of microcarriers said to be “edible”, which were specially developed for cultured meat applications involves chemical modifications of natural biomaterials (see e.g. WO-A-2015038988 discussed further below). An additional limitation is the low yield of the separation process of the cells from most of the existing microcarriers when microcarriers are not integrated with the cultured meat product. In contrast, degradable/dissolvable microcarriers were developed to provide a high yield of cell separation. In the category of degradable/dissolvable microcarriers, mainly ionically crosslinked polysaccharides through cations are used. However, the known methods used to increase the low stability of ionically crosslinked microcarriers are either not compatible with the food process, such as the use of toxic barium cations instead of calcium cations, or the known methods form microcarriers that can only be degraded using lysing enzymes. In the latter case, the enzymatically broken-down polysaccharide cannot be recycled, which makes the process cost-inefficient. Therefore, there is a need to address the aforementioned technical drawbacks in existing technologies for producing fully edible, dissolvable, inexpensive microcarriers to efficiently cultivate anchorage-dependent cells in bioreactors and release the cells at the end of the cell growth with a high yield without the use of enzymes, for the production of cultured meat products, for example pork meat, beef meat, chicken meat, crustacea meat, fish meat.

WO-A-2020219755 describes a cultured meat or food product with more desirable flavor and texture, as well as methods and compositions for producing the same. Certain embodiments are directed to methods of producing stiffer, structured surfaces or scaffolds that mimic the extracellular matrix (ECM) and support the growth of myotubes that are interspersed with a scaffold component supporting fat cells (adipocytes) in vitro.

WO-A-2015038988 relates to edible microcarriers, including microcarrier beads, microspheres and microsponges, appropriate for use in a bioreactor to culture cells that may be used to form a comestible engineered meat product. For example, the edible microcarriers described may include porous microcarriers that may be used to grow cells (e.g., smooth muscle cells) and may be included with the cells in the final engineered meat product, without requiring modification or removal of the cells from the microcarriers. In a particular example, the edible microcarriers may be formed of cross-linked pectin, such as pectin-thiopropionylamide (PTP), and RGD-containing polypeptides, such as thiolated cardosin A. Methods of forming edible microcarriers, methods of using the edible microcarriers to make engineered meat, and engineered meat including the edible microcarriers are also described.

WO-A-2013016547 describes engineered meat products formed as a plurality of at least partially fused layers, wherein each layer comprises at least partially fused multicellular bodies comprising non-human myocytes and wherein the engineered meat is comestible. Also provided are multicellular bodies comprising a plurality of non-human myocytes that are adhered and/or cohered to one another; wherein the multicellular bodies are arranged adjacently on a nutrient-permeable support substrate and maintained in culture to allow the multicellular bodies to at least partially fuse to form a substantially planar layer for use in the formation of engineered meat. Further described herein are methods of forming engineered meat utilizing said layers.

WO-A-2020123876 describes systems and methods for producing food products including cultured food products. The cultured food products include sushi-grade fish meat, fish surimi, foie gras, and other food types. Various cell types are utilized to produce the food products and can include muscle, fat, and/or liver cells. The cultured food products are grown in pathogen-free culture conditions without exposure to toxins and other undesirable chemicals. The food products can be processed to provide a desired shape, texture and consistency.

US-A-2016227831 describes dehydrated, edible, high-protein food products formed of cultured muscle cells that are combined (e.g., mixed) with a hydrogel (e.g., a plant-derived polysaccharide). These food products may be formed into a chip (e.g., snack chip), that has a protein content greater than 50%. One or more flavorants may also be included.

WO-A-2014025312 describes a method of manufacturing hydrogel microparticles comprising one or more species of living cells attached thereon and/or encapsulated therein. The method includes dissolving a hydrogel-forming agent in an aqueous medium to form a solution; suspending one or more species of living cells in the solution to form a cell suspension; dispersing the cell suspension into an organic oil to form a microemulsion; and subjecting the microemulsion to conditions that allow the hydrogel-forming agent to form hydrogel microparticles comprising one or more species of living cells attached thereon and/or encapsulated therein. Composition comprising a mixture of a degradable hydrogel and at least one hydrogel microparticle having one or more species of living cells, and method of manufacturing a scaffold for tissue engineering are also provided.

CN-A-113684177 discloses a composite microcarrier for manufacturing cell culture meat and a preparation method thereof. The composite microcarrier comprises microcarrier balls, the surfaces of the microcarrier balls are of a net-shaped structure, the microcarrier balls comprise polysaccharide, aldehyde acid and protein, the molar percentage of the polysaccharide to the aldehyde acid to the protein is (70-80%): (15-25%): (3-5%), a surface treatment layer is arranged on the surface of each microcarrier ball, the surface treatment layer comprises protein and polylysine, and the molar ratio of the protein to the polylysine is (35-90%): (10-20%). According to the invention, adherent myoblasts can be promoted to rapidly enter a differentiation stage, and then the myoblasts are promoted to realize a myogenic function.

### SUMMARY OF THE INVENTION

It is an object of the present invention to provide for new and improved microcarriers suitable and adapted to efficiently cultivate anchorage dependent cells in bioreactors. It is further an object of the present invention to propose methods to produce such microcarriers as well as the uses of such microcarriers.

A particular advantage of the present invention is to provide fully edible, dissolvable, inexpensive microcarriers to efficiently cultivate anchorage-dependent cells in bioreactors and preferably also to release the cells at the end of the cell growth with a high yield without the use of enzymes, for the production of cultured meat products, for example pork meat, beef meat, chicken meat, crustacea meat, fish meat.

The present invention provides fully edible, inexpensive microcarriers to efficiently cultivate cells in bioreactors and release the cells at the end of the cell growth with a high yield without the use of enzymes. In this invention, an edible mechanical stability agent is used to enhance the stability of microcarriers made of a ionically crosslinked polysaccharide that, preferably alone, slowly degrades when used in cell culture conditions. In a certain range of content of mechanical stability agent, the microcarriers may be dissolved by a chelator to release the cells. The microcarrier dissolution being mediated without lysis enzymes, the material of the microcarrier core can be recycled to cost-efficiently produce new microcarriers or hydrogel scaffolds. Moreover, often cells are not or to a lesser degree detached from the biopolymer coating material itself or from the extracellular matrix secreted by the cells over the cell growth. Cells may thus carry these materials with them when they are, for example, subsequently seeded on a scaffolding biomaterial. An improved and faster attachment on the scaffold may be thus achieved. One advantage of the microcarrier being edible is that either the microcarrier as such can remain in the food product to be eaten, or, if the microcarrier is removed, corresponding removal and purification processes do not need to be a high standard because it is no problem if elements of the microcarrier remain in the food product to be eaten.

The present disclosure presents microcarriers with easily tunable compositions. For example, the at least one coating can be easily changed from one biopolymer to another. Moreover, as the coating biopolymer, which has cell-binding properties, is often the most expensive ingredient of the microcarrier or might be derived from animal sources, it is very suitable to have a cheap microcarrier core and the cell-binding material only present in the very thin coating, minimizing thus the amount of biopolymer needed. Microcarrier stiffness may also be easily modulated to adapt to different cell types, e.g. by adjusting the alginate concentration in the microcarrier core.

The present disclosure provides edible microcarriers composed only of components safe for human consumption. Moreover, the microcarrier production process can be completely food safe. The microcarriers or parts of the microcarriers can thus be integrated into the cultured meat (CM). Filtration of microcarriers or parts of the microcarriers is not required to ensure food safety of the CM. Further, the texture of CM can be modulated for example through agar/agarose and alginate contents.

The present disclosure makes it possible to include high cell content (component D) in the microcarriers core in particular when using agarose or a low concentration of agar (<0.3%) as component B as a mechanical stability agent. Co-culturing by adding supporting cells or fat cells in the microcarrier core can improve the growth of cell cultured on the microcarrier surface. Moreover, the cell density in CM increases when the cell-loaded microcarriers are integrated into the final product. Further, co-culturing fat cells (e.g. adipocytes) in the microcarrier core and muscle stem cells on the surface, and incorporating said microcarriers in the CM may replicate the texture/composition of marbled meat.

The term “cultured meat” refers to food products that comprise animal-sourced cells produced by in vitro culture.

“Ionically crosslinked polysaccharide” describes here a water-loaded gel formed by a polysaccharide that gels through interactions with divalent and trivalent cations. “Ionically cross-linkable polysaccharides” are thus polysaccharides that can gel via an egg-box mechanism such as alginates and pectins or via helix-helix aggregation, for example, carrageenans. “Pectin” here always preferably refers to low methoxyl pectin unless stated otherwise.

The term “biopolymer” according to the general IUPAC definition here stands for macromolecules (including proteins, nucleic acids and polysaccharides) formed by living organisms, or derivatives thereof. Preferred embodiments of biopolymer are detailed further below. The biopolymer in the coating in this context is preferably used for cell-binding only, and it can be chosen so to bind the type of cells which are to adhere to the coating. The biopolymer can also be a RGD-polysaccharide layer around the core. Coating systems can be based on proteins but also can be based on polysaccharides with peptides and/or proteins attached or combinations thereof.

The term “mechanical stability agent” refers here to an edible component that is added to enhance the stability of the microcarrier core for example to resist the environment of a stirred tank bioreactor without being degraded or damaged. The mechanical stability agent may also alter the stiffness of the microcarrier core. The mechanical stability agent is preferably an edible component (B as defined further below) that gelles during or after the formation of the microcarrier core, i.e. during or after the ionic crosslinking of the main component (A as defined further below) in the bath. The mechanical stability agent Component (B) preferably gelles through a different mechanism than component (A), e.g. through thermal gelling, pH change (e.g. chitosan), covalent crosslinking (using a crosslinker such as glutaraldehyde or transglutaminase), or a combination thereof. The stability of the mechanical stability agent gelled state should be suitable to the cultivation conditions (temperature, pH, stirring, etc). So, it should typically be stable at pH 6-5-8, preferably in the range of 7-7.5, it should typically not be ionically crosslinked, it should typically be stable at 37° C. Preferably, mechanical stability agent is chosen to be a thermally-responsive polysaccharide. The stability of the microcarrier can be adjusted by the concentration and type of the mechanical stability agent used. For example, if the goal is to dissolve the microcarriers after the cell growth, low agent concentration will be used. If the microcarriers will end up in the final product, a higher concentration can be used. The absence of a stability agent in an ionically crosslinked microcarrier typically makes it too unstable and thus would not perform well for cell culture, as the coating is not protective enough.

A “thermo-responsive polysaccharide” means a polysaccharide that exhibits an upper critical solution temperature (UCST), transitioning from a solution to a gel state upon cooling or a lower critical solution temperature (LCST), transitioning from a solution to a gel state upon heating. To work as a mechanical stability agent, the polysaccharide should preferably be in a gel state at the temperature, in which cells are cultured, typically between 10-40° C., depending on the cell type and species source, i.e. it preferably should have a UCST above the culturing temperature and/or a LCST below the culturing temperature. Moreover, it preferably should be possible to obtain the liquid state of the thermo-responsive polysaccharide in a temperature range compatible with the processability conditions of the other components of the microcarrier core.

Agar, agarose, carrageenan, xyloglucan, methylcellulose, gellan gum or combinations thereof are thermo-responsive polysaccharides (certain xyloglucan types may require a modification to become thermoresponsive). Also possible are mixtures of two or more non-thermo-responsive polysaccharide that become thermo-responsive when mixed. Note that carrageenan gels upon cooling. So because carrageenan gels thermally, a microcarrier using carrageenan as component A (ionically crosslinked polysaccharide) is not dissolvable using a chelator, one would have to heat it too, which is not compatible with cell culture. Therefore carrageenan can normally only be used as a mechanical stability agent and is not used as ionically crosslinked polysaccharide.

The protein coating is preferably cross-linked, and further preferably “covalently cross-linked”, meaning here that the protein network is crosslinked together. The statement does not necessarily describe a crosslinking of the microcarrier coating to the microcarrier core unless the microcarrier also incorporates a protein in its core. Cross-linking may be a covalent cross-linking but also or in addition to supramolecular cross-linking.

A “degradable microcarrier” refers here to a microcarrier whose constituent polymer chains of the microcarrier core can be broken down to an extent that allows cells cultured on the microcarrier surface to be released and harvested. The degradation is mostly mediated through enzymes to prevent affecting the cell viability.

A “dissolvable microcarrier” refers here to a microcarrier of which the crosslinking of the constituent polymer network of the microcarrier core can be reversed to an extent that allows cells cultured on the microcarrier surface to be released and harvested. The integrity of the polymer chains is thus not affected by the dissolution process.

“Supporting cells” refers here to cells that are known to secrete cell-modulating factors, e.g. growth factors and that can thus support the growth of other cells, e.g. stem cells. The term “polyanionic polysaccharide,” also as used herein, is intended to designate polymeric polysaccharides and their derivatives containing anionic groups at physiological pH.

A first aspect of the present disclosure relates to an edible microcarrier for growing anchorage-dependent cells to prepare a cultured meat product. The edible microcarrier has a microcarrier core and a microcarrier coating. The microcarrier core is preferably a hydrogel that comprises 0.03-10.0 wt % of ionically crosslinked polysaccharide, 0.05-5 wt % of a mechanical stability agent and water. The microcarrier coating comprises a biopolymer. The biopolymer is crosslinked using a crosslinking agent.

Said microcarrier core is a hydrogel consisting of the following components:
(A) 0.03-10.0 wt % of at least one ionically crosslinked polysaccharide;
(B) 0.05-5 wt % of a mechanical stability agent different from (A);
(C) 0.0-10 wt % of additives different from (A) and (B);
(D) 0.0-50 wt % living cells;
(E) 75-99.92 wt % of water;
the sum of (A)-(E) making up 100 wt %, and
wherein said microcarrier coating comprises or consists of a biopolymer and the biopolymer is preferably crosslinked using a crosslinking agent.

The main gist of the proposed edible microcarriers is the use of the edible mechanical stability agent allowing to improve the stability of ionically crosslinked microcarriers while still allowing them to dissolve easily when required. The aim of the microcarrier coating is not primarily or even not at all to protect the microcarrier core. According to the invention the microcarrier stability is enhanced by the mechanical stability agent in the microcarrier core, so that the microcarrier stability is ensured whatever microcarrier coating is used. The microcarrier coating's main role is cell attachment, not to protect the core, because the latter is already ensured by the microcarrier core itself and the mechanical stability agent, which provides thus more flexibility on the coating composition, process, etc. to adapt to the cell types and culturing conditions.

Between the microcarrier core and the microcarrier coating or as part of the latter a primer coating can be provided, i.e. an intermediary coating between the microcarrier core and the biopolymer cell-adhesive coating. Said primer coating may be used to enhance the adhesion of the cell-binding coating. Preferably the primer coating is selected from a protein, a polypeptide or a polysaccharide or a combination thereof, more preferably the primer coating is based on or consists of chitosan, chitin, polylysine or a combination thereof.

According to a preferred embodiment, component (A) in said microcarrier core is a ionically crosslinked polyanionic polysaccharide, preferably selected from the group consisting of: alginate, pectin, hyaluronan, gum arabic, xanthan gum and carboxymethylcellulose, carboxymethyl amylose, carboxymethyl chitosan, chondroitin sulfate, dermatan sulfate, heparin, heparin sulfate, and any of their salts, including sodium, potassium, magnesium, calcium, ammonium, or mixtures thereof and/or derivatives of any of these, more preferably alginate, pectin, or a combination thereof.

Typically, these component (A) systems have a molecular weight in the range of 50-3000 kDa, preferably in the range of 80-1000 kDa.

Component (B) of the mechanical stability agent in said microcarrier core is preferably selected from a thermo-responsive polysaccharide, preferably selected from the group consisting of: agar, agarose, carrageenan, xyloglucan, methylcellulose, gellan gum or mixtures thereof and/or derivatives of any of these, more preferably from agar. Typically, these mechanical stability agent systems have a molecular weight in the range of 50-3000 kDa, preferably in the range of 80-1000 kDa.

Component (C) in said microcarrier core is preferably selected from the group consisting of: osmolarity regulating compounds; sugar alcohols; protein, preferably, selected from gelatin, collagen, dietary fibres, fibrinogen, fibrin, fibronectin, elastin, laminin, soy protein, zein protein, pea protein, canola protein, carob protein, cardosine A, wheat protein, albumin, casein protein, potato protein, guar protein, protein hydrolysates, or a combination thereof, wherein the protein can be crosslinked using said crosslinking agent (which preferably is transglutaminase, peroxidase, laccase, tyrosinase, lysyl oxidase, glutaraldehyde, genipin, citric acid, tannic acid or a combination thereof, preferably from transglutaminase, genipin, glutaraldehyde or a combination thereof, in particular transglutaminase); mono- and oligosaccharides including natural mixes such as corn syrup, honey, maple syrup, glucose syrup; or mixtures thereof and/or derivatives of any of these, preferably selected from the group consisting of an osmolarity regulating compound of a monosaccharide, a disaccharide, an oligosaccharide, or a combination thereof, or mixtures thereof and/or derivatives of any of these.

Component (D) is preferably muscle cells, fat cells, connective cells, fibroblasts, pericytes or a combination thereof.

Said microcarrier core further preferably comprises component (A) in an amount of 0.5-7.5 wt %, preferably of 0.7-5.5 wt %, more preferably of 0.8-4.5 wt % or 0.8-3.5 wt %.

Said microcarrier core preferably comprises component (B) in an amount of 0.1-3.5 wt %, preferably of 0.15-1.5 wt %, more preferably of 0.17-0.35 wt %.

Said microcarrier core preferably comprises component (C) in an amount of 0.01-7.5 wt %, preferably of 0.1-5 wt %, more preferably of 1-4 wt %.

Said microcarrier core preferably comprises component (D) in an amount of 0.00-40 wt %, preferably of 0.1-25 wt %, more preferably of 1-10 wt %.

In each case, the complement such that the sum of (A)-(E) is making up 100 wt %, is given by component (E).

Component (A) is preferably crosslinked by salt cations selected from the group consisting of: Ca2+, Mg2+, Fe2+ and Fe3+ or a combination thereof, preferably by salt cations of Ca2+.

According to yet another preferred embodiment, said biopolymer in the microcarrier coating is a protein (at least 100 amino acids) or a polypeptide (built from at least 10 and less than 99 amino acids or at least 20 and less than 99 amino acids) or a polysaccharide, preferably selected from the group consisting of: gelatin, collagen, fibrinogen, fibrin, fibronectin, elastin, laminin, soy protein, zein protein, pea protein, canola protein, carob protein, cardosine A, wheat protein, albumin, casein protein, potato protein, guar protein, or a combination or derivative thereof more preferably from gelatin and derivative thereof.

Preferably, if the coating protein is positively charged at pH between 6-8, the microcarrier coating is applied directly on the microcarrier core.

Further preferably, if the coating protein is negatively charged at pH between 6-8, a positively charged primer coating is applied first, wherein the primer coating is selected from a protein, a polypeptide or a polysaccharide or a combination thereof, more preferably from chitosan, chitin, polylysine or combination thereof.

Said crosslinking agent can be selected from the group consisting of: transglutaminase, peroxidase, laccase, tyrosinase, lysyl oxidase, glutaraldehyde, genipin, citric acid, tannic acid or a combination thereof, preferably from transglutaminase, genipin, glutaraldehyde or a combination thereof, more preferably from glutaraldehyde or transglutaminase, in particular glutaraldehyde.

The edible microcarrier is preferably dissolvable using a chelator, wherein the chelator is preferably EDTA or sodium citrate. Dissolution is preferably done with a chelator concentration of 1-100 mM within 1-90 min at a temperature between 4 and 40° C.

So the edible microcarriers may be dissolvable using only inexpensive food-grade chelators to cost efficiently release the cells at the end of proliferation with a high yield and without the use of enzymes. As the microcarrier components are approved for food consumption, no extensive filtration process is required to filter out the dissolved microcarriers materials. It is also possible to recycle the dissolved microcarrier materials as the microcarriers are dissolved without degrading the materials by enzymes. The cell release from commercially available microcarriers most often requires the use of expensive and less cell-friendly proteolytic enzymes (e.g. Trypsin). Some existing microcarriers were developed to be dissolved after completion of cell culture to release the cells using non-proteolytic enzymes (e.g. alginate lyase or pectinase) and a chelator (e.g. EDTA), however, these microcarriers are not edible and the microcarrier materials cannot be recycled.

The possible use of chelators shows that the proposed microcarrier cores are superior as no enzymes are needed to dissolve them, thus the polymer of the core is not degraded and can be recycled.

As pointed out above, a “dissolvable microcarrier” refers to a microcarrier of which the crosslinking of the constituent polymer network of the microcarrier core can be reversed to an extent that allows cells cultured on the microcarrier surface to be released and harvested. To properly dissolve the microcarrier, it is preferable to degrade the microcarrier core and microcarrier coating too, for example in the case where the coating protects the core from the chelator, as a barrier. To this end preferably a mixture of an enzyme and a chelator, preferably collagenase and EDTA is used, to dissolve the microcarriers. Collagenase is an enzyme degrading specifically the coating, EDTA is a chelator dissolving specifically the core.

It is important to note, as pointed out above, that the mechanical stability agent is required for the microcarrier to perform well over time in media. Without the mechanical stability agent, the microcarriers swell and slowly degrade. So the coating may indeed protect the core to some extent (for example from a chelator such as EDTA during the dissolution), but typically cannot replace the role of the mechanical stability agent namely inter alia to prevent the microcarrier from degradation in media.

Since the enzyme preferably degrades only the coating, the core material is only dissolved by the chelator and can still be recycled. For example, it was found that one can dissolve the microcarrier by adding EDTA/collagenase to release the cells, then one can remove EDTA/collagenase from the solution where cells and dissolved alginate remain, finally one can directly inject dropwise this solution into a crosslinking bath and form beads where cells are encapsulated inside it. Afterwards one can proceed to the cell differentiation phase to form e.g. fat tissue in the form of beads.

The microcarriers may include high cell content in the microcarrier core (component D). Co-culturing by adding supporting cells or fat cells in the microcarrier core may increase the proliferation of stem cells growing on the microcarrier surface. Moreover, the cell density in the cultured meat (CM) increases when the cell-loaded microcarriers are integrated into the final product. Further, the texture/composition of marbled meat can be replicated by co-culturing fat cells (e.g. adipocytes) in the microcarrier core and muscle stem cells on the surface and incorporating the microcarriers in the CM.

The edible microcarrier may be a spherical, cylindrical, fiber-shaped, ovoidal, or irregular shape. The microcarrier core has typically one dimension comprised between 0.05 mm and 2 mm.

As mentioned, optionally, the microcarrier core further comprises an osmolarity regulating compound, wherein the osmolarity regulating compound is preferably present in an amount of 1-5 wt %, wherein the osmolarity regulating compound is preferably selected from a monosaccharide, a disaccharide, an oligosaccharide, or a combination thereof.

The microcarrier core may further comprise a protein, preferably, the protein in the microcarrier core is selected from gelatin, collagen, fibrinogen, fibrin, fibronectin, elastin, laminin, soy protein, zein protein, pea protein, canola protein, carob protein, cardosine A, wheat protein, albumin, casein protein, potato protein, guar protein, or a combination thereof. A concentration of the protein is preferably comprised in an amount of 0.05-5 wt %. The protein can be crosslinked using the crosslinking agent to enhance the stability of the microcarrier protein coating to the microcarrier core.

If the coating protein is negatively charged at pH between 6-8 a positively charged primer coating can be applied first. The primer coating can be selected from a protein, a polypeptide, or a polysaccharide. The primer coating is used as an intermediary layer between a negatively charged microcarrier core and a negatively charged polymer coating. The primer coating can be made of a positively charged polymer to optimize the interactions with the microcarrier core and the microcarrier coating. For example, the primer coating can be selected from a positively charged protein, a polypeptide or a polysaccharide, or a combination thereof. The formation of the microcarrier coating can be through electrostatic interactions between the negatively charged microcarrier core and the positively charged coating biopolymer.

Optionally, the edible microcarrier additionally comprises supporting cells, in the microcarrier core, wherein the supporting cell is selected from fibroblasts or pericytes. The supporting cells which increase the proliferation of cells on the surface of the microcarrier by producing specific factors including but not limited to growth factors required for proliferation and/or differentiation of cells may be immobilized in the microcarrier and thus presented to the cells more efficiently compared to their addition in the cultured media.

A second aspect of the present disclosure provides a method of producing edible microcarriers, preferably as described above, for growing anchorage-dependent cells to prepare a cultured meat product.

The method comprises the steps of:
a) obtaining a precursor hydrogel solution of microcarrier core, preferably in the form of a solution consisting of the following components:
(A) 0.03-10.0 wt % of at least one ionically cross-linkable polysaccharide;
(B) 0.05-5 wt % of a mechanical stability agent different from (A), or, if component (A) at the same time acts as a mechanical stability agent, 0.0-5 wt % of a mechanical stability agent different from (A);
(C) 0.0-10 wt % of additives different from (A) and (B);
(D) 0.0-50 wt % living cells;
(E) 75-99.92 wt % of water;
the sum (A)-(E) making up 100 wt %;
b) forming droplets from said precursor hydrogel solution;
c) ionically crosslinking the droplets in a gelling solution through interactions with salt cations to form said microcarrier core;
d) preferably keeping the microcarrier core immersed in a solution, preferably containing 0.05%-3%, of a coating biopolymer to produce said coated edible microcarrier; before applying the coating in step d) or as part of step d) a primer coating can be applied to the core; and
e) optionally cooling down the edible microcarrier for thermal gelling of the microcarrier core.

In steps a) and b) the temperature of the solution can be controlled in the range between 20° C. and 85° C., preferably in the range between 55° C. to 80° C.
f) optionally immersing the microcarrier in a solution containing a crosslinking agent to cross-link the coating biopolymer.

A washing step between coating in step d) and final crosslinking in step f) is possible. The concentration of the salt cations in said gelling solution can be comprised in the range between 20 mM and 1 M, wherein preferably the salt cations in said gelling solution are selected from Ca2+, Mg2+, Fe2+ and Fe3+ or a combination thereof, preferably from salt cations of Ca2+.

Before or after step (e) said coated edible microcarrier can be immersed in a crosslinking bath, preferably comprising crosslinking agent for the coating biopolymer in an amount of 1-20 or 5-10 mg/ml preferably for a time span in the range of 1-300 minutes, or 50 minutes to 120 minutes.

Preferably the crosslinking agent is selected from transglutaminase, peroxidase, laccase, tyrosinase, lysyl oxidase, glutaraldehyde, genipin, citric acid, tannic acid or a combination thereof, preferably from transglutaminase, genipin, glutaraldehyde or a combination thereof, more preferably from glutaraldehyde or transglutaminase, in particular glutaraldehyde. If not already adhered, cells in such a process can generally be grown and/or adhered around the microcarrier to the microcarrier coating, and subsequently the edible microcarrier, preferably the microcarrier core and/or the microcarrier coating, is dissolved and/or degraded using a chelator and/or an enzyme, preferably a combination of an enzyme and a chelator. The chelator is preferably EDTA or sodium citrate and dissolution is preferably done with a chelator concentration of 1-100 mM within 1-90 min at a temperature between 4 and 40° C. The enzyme is preferably an enzyme degrading the biopolymer of the coating, preferably selected to be collagenase, subsequently chelator and/or enzyme are removed from the solution and this solution is cross-linked, preferably by introducing into a cross-linking bath, to form beads with the cells encapsulated inside the previously dissolved microcarrier core material, preferably followed by cell differentiation to form the desired tissue.

The droplets of hydrogel solution may be formed using a device based on microfluidic droplet formation, electrostatic droplet formation, a vibration jet breakage device, a rotating jet breakage device, a co-axial airflow device, simple dropping, or water-in-oil emulsion.

A third aspect of the present disclosure provides the use of the edible microcarrier for the preparation of a cultured meat product.

A fourth aspect of the present disclosure provides cultured meat product comprising such an edible microcarrier.

Further embodiments of the invention are laid down in the dependent claims.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows a schematic illustration of an edible microcarrier for growing anchorage-dependent cells to prepare a cultured meat product according to an embodiment of the present disclosure;

FIG. 2 shows a schematic illustration of a coaxial flow system for producing edible microcarriers for growing anchorage-dependent cells to prepare a cultured meat product according to an embodiment of the present disclosure;

FIG. 3 shows flowcharts illustrating a method for producing edible microcarriers for growing anchorage-dependent cells to prepare a cultured meat product according to an embodiment of the present disclosure;

FIG. 4 shows primary satellite cells isolated from a Limousin calf grown 9 days on gelatin-coated alginate-agar microcarriers in spinner flasks;

FIG. 5 shows primary pre-adipocytes isolated from a Wagyu cow proliferated 3 days on gelatin-coated alginate-agar microcarriers in a well plate; and

FIG. 6 shows cells released after the dissolution of the microcarriers by treatment in 5 mM EDTA.

### DESCRIPTION OF PREFERRED EMBODIMENTS

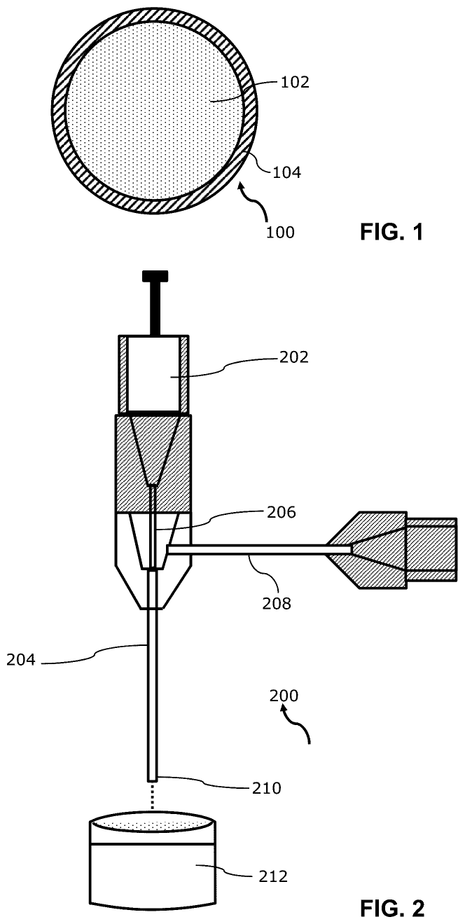

FIG. 1 is a schematic illustration of an edible microcarrier 100 for growing anchorage-dependent cells to prepare a cultured meat product according to an embodiment of the present disclosure. The edible microcarrier is a hydrogel that includes a microcarrier core 102 and a microcarrier coating 104. The microcarrier core 102 is made of or comprises 0.3-10.0 wt % of an ionically cross-linked polysaccharide, 0.05-5 wt % of a mechanical stability agent, if needed further additives and water. The microcarrier coating 104 includes a biopolymer. The biopolymer is cross-linked using a cross-linking agent.

In an example embodiment, edible, dissolvable microcarriers are produced in a completely food-safe process using a co-axial airflow bead-making setup. FIG. 2 is a schematic illustration of a coaxial flow system 200 for producing edible microcarriers for growing anchorage-dependent cells to prepare a cultured meat product according to an embodiment of the present disclosure. The coaxial flow system 200 includes a syringe pump system 202, a coaxial needle 204, an inner inlet 206, an outer inlet 208, a coaxial needle outlet 210, a gelling bath 212. A hydrogel solution of microcarrier core precursors comprising 0.3-10.0 wt % of an ionically cross-linkable polysaccharide, 0.05-5 wt % of a mechanical stability agent, and water are loaded in the syringe pump system 202. The syringe pump system 202 comprising the hydrogel solution of microcarrier core precursors is heated to a temperature between 20° C. to 85° C. The heated hydrogel solution is provided into a coaxial needle 204. The heated hydrogel solution is provided through the inner inlet 206 and filtered air is passed through the outer inlet 208 of the coaxial needle 204. The gelling bath 212 is prepared to—contain 20 mM-2M of salt cations of Ca2+, Mg2+, Fe2+ or Fe3+, or a combination thereof, optionally 0.05%-3% of coating agent, and optionally 1 mM-50 mM of HEPES. A flow rate of the hydrogel solution and air pressure of the filtered air entering through the outer inlet 208 of the coaxial needle 204 is adjusted to facilitate falling of droplets of the hydrogel solution through the coaxial needle outlet 210 in the gelling bath 212. The droplets are cross-linked by the salt cations of the gelling bath 212 to form a microcarrier core. The microcarrier core is coated with a coating biopolymer. The coated microcarrier core is immersed in a crosslinking bath comprising a crosslinking agent for about 5 minutes to 1000 minutes. The edible microcarriers are obtained for the preparation of a cultured meat product.

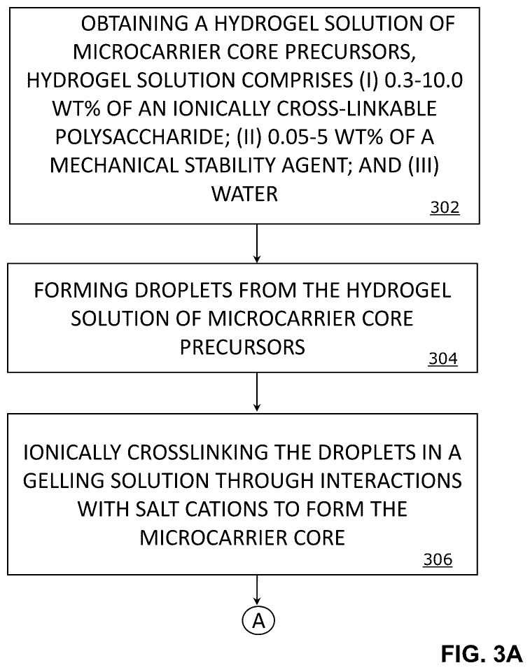

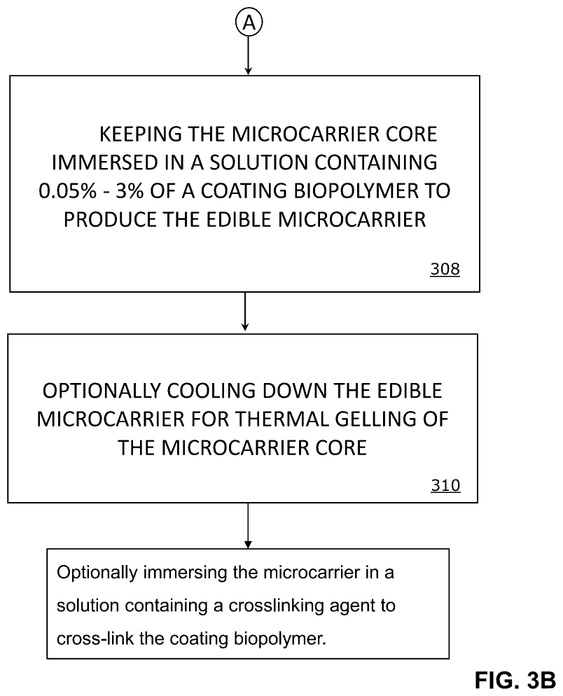

FIGS. 3A and 3B are flowcharts illustrating a general method for producing edible microcarrier for growing anchorage-dependent cells to prepare a cultured meat product according to an embodiment of the present disclosure. At a step 302, a hydrogel solution of microcarrier core precursors is obtained, the microcarrier core precursors comprise (i) 0.3-10.0 wt % of an ionically cross-linkable polysaccharide; (ii) 0.05-5 wt % of a mechanical stability agent; and (iii) water. At a step 304, droplets from the hydrogel solution of microcarrier core precursors is formed. At a step 306, the droplets are ionically crosslinked in a gelling solution through interactions with salt cations to form the microcarrier core. At a step 308, the microcarrier core is kept immersed in a solution containing 0.05%-3% of a coating biopolymer to produce the edible microcarrier. At a step 310, the edible microcarrier are optionally cooled down for thermal gelling of the microcarrier core.

So the production steps, in more detail, include:
obtaining a hydrogel solution of microcarrier core 102 precursors of 2.5% sodium alginate, 0.2% agar, 3% D-mannitol and water;
loading the hydrogel solution in a syringe pump system;
heating the syringe pump system 202 and comprising the hydrogel solution, and the co-axial needle 204 between 60° C. to 75° C.;
providing the heated hydrogel solution to the inner inlet 206 of the co-axial needle 204;
adjusting the flow rate of the hydrogel solution between 0.5-8 ml/min or 0.5-1 ml/min and the air flow pressure connected to the outer inlet 208 of the co-axial needle 204 between 0.4-1.4 bar to obtain an appropriate formation of droplets falling into a gelling bath 212;
crosslinking the droplets in the gelling bath 212 containing 100 mM calcium chloride salt and normally additionally, 5 mM HEPES to form a hydrogel microcarrier core 102;
keeping the microcarrier core 102 immersed in the gelling bath 212 containing also 0.3%-1.0% gelatin type A to initiate the formation of the microcarrier coating 104 through electrostatic interactions between the negatively charged microcarrier core 102 and the positively charged gelatin chains;
immersing the coated microcarrier core 102 in a crosslinking bath comprising transglutaminase in an amount of 5-10 mg/ml or 0.05%-0.5% glutaraldehyde for about 50 minutes to 120 minutes;
obtaining the edible microcarrier 100 for the preparation of a cultured meat product.

Specific and preferred values disclosed for components, ingredients, conditions, parameters, and ranges thereof, are broader than the ones specified in the examples. For example, these values can be greatly influenced by the specific ingredient or combination of ingredients selected, the ingredient source, the molecular weight, the type of production device (e.g. droplet generator), conditions used, and/or possible post-treatments. Lyophilization is an example of post-processing that can greatly affect the polymer content of the hydrogel microcarrier 100. For example, a batch of microcarriers 100 produced from hydrogel solution comprising 2.5% sodium alginate, 0.2% agar, 3% D-mannitol, and water can be weighted after their production in a wet state, and same after a freeze-drying and re-wetting cycle. The dry mass content of the microcarrier increased 2-fold over the process. For example, the microcarrier 100 produced from hydrogel solution comprising 2.5% sodium alginate, 0.2% agar, 3% D-mannitol, and water is obtained in a dimension ranging from about from 100 to 350 μm in diameter. The molecular weight of sodium alginate is comprised in the range between 120-190 kDa. Rheological measurements of bulk hydrogels of the same composition were performed by small-amplitude oscillatory shears (SAOS) at a frequency of 1 Hz. SAOS was used to measure values of storage shear modulus at 37° C. Stiffness was calculated from the storage shear modulus by assuming a Poisson's ratio of 0.5. The resulting stiffness at 37° C. corresponds to 193.7+4.2 kPa just after the crosslinking. Whereas the stiffness drops down to 120.8+2.5 kPa when hydrogels are stored for 24 h in culture conditions, probably due to the partial substitution of Ca2+ in the alginate network by non-crosslinking Na+ cations as well as due to the partial Ca2+ sequestration by free amino acids present in the media due to their chelation ability. Before use, the microcarriers may be stored in liquid suspension, or they may be freeze-dried/lyophilized. For the latter option, prior use of edible microcarriers should be rehydrated in salt cations of Ca2+ (20 mM-2M concentration range) for 30 sec-2 h.

The stability of the microcarrier in a bioreactor environment increases with the presence of a mechanical stability agent in the microcarrier core. For example, sodium alginate or pectin microcarriers crosslinked by Ca2+ cations are not stable enough to sustain the dynamic environment of a bioreactor filled with culture media. The liaison between alginate chains and Ca2+ cations is weak. Low content of Ca2+ cations and the presence of Na+ in the media promote the substitution of crosslinking Ca2+ cations by non-crosslinking Na+ in the microcarrier core, rendering the stability of the microcarrier core fragile. Instead of additional non-food-grade crosslinking of the alginate network, microcarriers can be reinforced for example by adding a thermo-responsive polysaccharide. After gelling upon cooling, the thermo-responsive polysaccharide may provide a more stable double network with the alginate. Microcarriers comprising no agar in the core still showed apparent degradation and breakage after being incubated in a stirring bioreactor in media for 3 days, while microcarriers with an agar content (A1296, Sigma) in particular above 0.15% were still intact after 25 days.

In an example embodiment, the amount of agar in the microcarrier core is used to tailor the dissolvability properties of the microcarrier by chelators. In a certain range of agar content, the stability of alginate microcarrier core improves compared to agar-free microcarriers while still being dissolvable in the presence of a chelating agent. Such properties are very convenient when microcarriers are not directly incorporated in the cultured meat product and cells must thus be released at the end of proliferation for example by dissolving the microcarrier

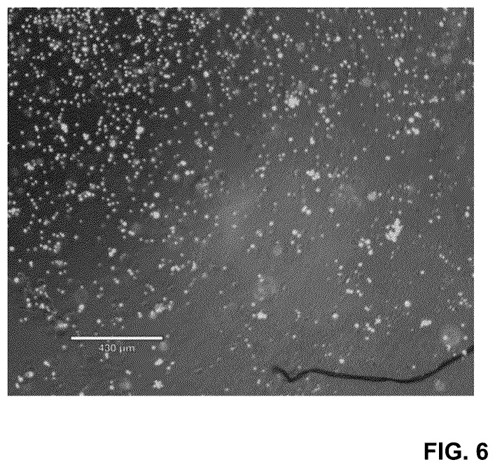

FIG. 6 shows dissolved microcarriers after treatment in 5 mM EDTA. The Cells are stained using Calcein-AM live staining and viewed under Fluorescence microscopy, FITC (LED) having an inverted setup with 4× Magnification.

The microcarriers produced from a hydrogel solution containing 2.5% sodium alginate, 0.2% agar, 3% D-mannitol, and water are dissolved by adding 5 mM EDTA to the media, in 15-25 min at 37° C., with a stirring speed of 100-140 rpm, ensuring high cell viability. The dissolution of a microcarrier is possible for a microcarrier produced from a hydrogel solution containing for an agar (A1296, Sigma) content of 0%, 0.05%, 0.10%, 0.15%, 0.20%, 0.25%, 0.30% and 0.35% and all the values comprised in between. Using this specific source of agar, higher agar content does not allow the dissolution of the microcarrier using a chelating agent alone. On this other side, below 0.15 wt % the microcarrier was mechanically less stable and below 0.05 wt % the microcarrier was mechanically not stable when incubated in media with stirring (100 rpm) as they slowly deformed and started to break already after 24 hours of incubation. One the other hand By adjusting the agar content, the texture of the microcarrier may be tuned. These properties are useful when the microcarrier is incorporated into the final cultured meat product.

The biopolymer coating allows the cells to attach and proliferate on the microcarrier. Both pre-adipocytes and satellite cells showed very poor attachment to microcarriers e.g. produced from a hydrogel solution containing 2.5% sodium alginate, 0.2% agar, 3% D-mannitol, and water, without biopolymer coating. Most cells did not interact at all with the microcarriers, and no proliferation was observed. On the other hand, when a coating is applied using e.g. a 0.5% gelatin A solution and crosslinked in 0.1% glutaraldehyde solution, cells attached very well and proliferated until covering the whole microcarrier surface. In an example embodiment, a protein is included in the microcarrier core 102 to enhance the stability of the microcarrier coating 104. For example, a microcarrier core 102 is produced using a co-axial airflow bead-making setup from a hydrogel precursors solution of 2.5% sodium alginate, 0.2% agar, 1% gelatin type B, 3% D-mannitol, and water and crosslinking the bead in a solution of 100 mM Ca2+. The microcarriers core 102 is then coated by immersion in a solution containing 0.5% gelatin type A. The subsequent crosslinking through transglutaminase or glutaraldehyde to crosslink the protein coating may also crosslink the coating to the protein (e.g. gelatin) included in the microcarrier core 102, thus enhancing the stability of the coating.

In an example embodiment, microcarriers, cells, and media are placed together in a spinner flask or a stirred tank bioreactor. During the seeding phase, the stirring is discontinuous to allow cells to attach to the microcarriers. Once cells are attached, the tank is stirred continuously to suspend the cell-loaded microcarriers homogeneously in the media for cell proliferation.

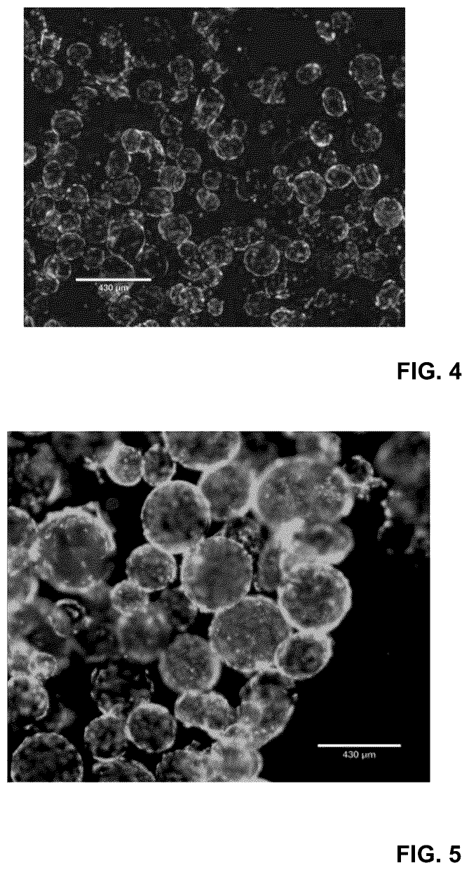

FIG. 4 shows primary satellite cells isolated from a Limousin calf grown 9 days on gelatin-coated alginate-agar microcarriers in spinner flasks. The cells are stained using Calcein-AM live staining and viewed under Fluorescence microscopy, FITC (LED) having an inverted setup with 4× Magnification.

FIG. 5 shows primary pre-adipocytes isolated from a Wagyu cow proliferated 3 days on gelatin-coated alginate-agar microcarriers in a well plate. The cells are stained using Calcein-AM live staining and viewed under Fluorescence microscopy, FITC (LED) having an inverted setup with 4× Magnification.

The mechanical stability agent, for example, 0.2% agar, enables microcarriers to be stable for a long incubation time in a stirred tank (up to 25 days).

In an example embodiment, crosslinking the gelatin coating of the microcarriers using transglutaminase or glutaraldehyde allow a good cell attachment or proliferation when being cultured in a spinner flask. Microcarriers 100 with a core 102 made of 2.5% sodium alginate, 3% D-mannitol and 0.2% agar and with a non-crosslinked gelatin coating do allow only a low cell attachment. The attached cells then do not proliferate and detach from the microcarrier after a few days.

In an example embodiment, high cell density may be incorporated in the microcarrier core. For example, pre-adipocyte cells are suspended in a hydrogel solution containing 1% alginate, 1% agarose and media. Droplets are formed using a co-axial airflow bead-making setup. During the droplet formation, the temperature of the cell-containing solution is kept at 37° C. The droplets are gelled in a gelling bath containing 100 mM calcium chloride salt to obtain a cell-loaded microcarrier core. The microcarrier core is coated with gelatin and gelatin is crosslinked in a transglutaminase bath. Muscle stem cells are attached on the microcarrier surface. Finally, the edible microcarrier containing the muscle and fat cells are used for the preparation of cultured meat product with high cell density and a texture and composition like marbled meat.

In an example embodiment, the ionically crosslinked polysaccharide, and the mechanical stability agent of the microcarrier core may be recycled to produce new microcarriers. The dissolution of the microcarrier for the cell release at the end of the proliferation is mediated without the use of enzymes that lysis the polymer chains of the microcarrier core. It was shown for example that the alginate comprised in microcarrier used to entrap algae and dissolved in 0.5M sodium citrate can be recovered with a yield up to 70% at lab-scale (Murujew et al, DOI: 10.1080/09593330.2019.1673827). This resulted in a net operational cost reduction of about 60%.

In an example embodiment, the ionically crosslinked polysaccharide of the microcarrier core may be used to encapsulate the cells after the dissolution of the microcarriers. Cells were proliferated on microcarriers produced from a hydrogel solution containing 2.5% sodium alginate, 0.2% agar, 3% D-mannitol, and water and coated with gelatin A. After the cells proliferated, the microcarrier could be dissolved using 10 mM EDTA and collagenase. After centrifugation, a pellet of cells and dissolved microcarrier materials was obtained. The pellet was resuspended at high concentration in media and the solution was loaded in a syringe equipped with a needle. Droplets of the pellet solution were dropped in a solution of 100 mM calcium chloride, 10 mM HEPES to crosslink them and form cell-loaded gel beads.

Cells encapsulated in the beads can then be differentiated according to the needs (fat or muscle tissue).

In an example embodiment, microcarriers covered with muscle cells, fat cells, or stem cells, preferably selected from adipocytes, myoblasts, satellite cells, pre-adipocytes, MSCs, or a combination thereof, may be integrated into a hydrogel solution ink used for 3D bioprinting for the preparation of a structured piece of meat. The differentiation into muscle or fat cells may be performed before or after the printing process.

In an example embodiment, microcarriers covered with muscle cells, fat cells, or stem cells, preferably selected from adipocytes, myoblasts, satellite cells, pre-adipocytes, MSCs, or a combination thereof, are stacked in a mould. The microcarriers may then be enzymatically bonded together using transglutaminase to obtain a meat product.

LIST OF REFERENCE SIGNS

| | |
|---|---|
| 100 | edible microcarrier |
| 102 | microcarrier core |
| 104 | microcarrier coating |
| 200 | coaxial flow system |
| 202 | syringe pump system |
| 204 | coaxial needle |
| 206 | inner inlet |
| 208 | outer inlet |
| 210 | coaxial needle outlet |
| 212 | gelling bath |
| 302-310 | steps |

1. An edible microcarrier for growing anchorage-dependent cells to prepare a cultured meat product, wherein said edible microcarrier comprises a microcarrier core and at least one microcarrier coating at least partially directly or indirectly covering the surface of said microcarrier core,
wherein said microcarrier core is a hydrogel consisting of the following components:
(A) 0.03-10.0 wt % of at least one ionically crosslinked polysaccharide;
(B) 0.05-5 wt % of a mechanical stability agent different from (A), or, if component (A) at the same time acts as a mechanical stability agent, 0.0-5 wt % of a mechanical stability agent different from (A);
(C) 0.0-10 wt % of additives different from (A) and (B);
(D) 0.0-50 wt % living cells;
(E) 75-99.92 wt % of water;
the sum of (A)-(E) making up 100 wt %, and
wherein said microcarrier coating comprises:
a biopolymer, wherein the biopolymer is crosslinked using a crosslinking agent.

2. The edible microcarrier according to claim 1, wherein component (A) in said microcarrier core is a ionically crosslinked polyanionic polysaccharide.

3. The edible microcarrier according to claim 1, wherein component (B) in said microcarrier core is selected from at least one of a thermo-responsive protein and/or a thermo-responsive polysaccharide.

4. The edible microcarrier according to claim 1, wherein component (C) in said microcarrier core is selected from the group consisting of: osmolarity regulating compounds; sugar alcohols; protein, protein hydrolysates; mono- and oligosaccharides or mixtures thereof or derivatives of any of these.

5. The edible microcarrier according to claim 1, wherein said microcarrier core comprises
component (A) in an amount of 0.5-7.5 wt %;
and/or component (B) in an amount of 0.1-3.5 wt %;
and/or component (C) in an amount of 0.01-7.5 wt %;
and/or component (D) in an amount of 0.00-40 wt %;
wherein in each case the complement, such that the sum of (A)-(E) is making up 100 wt %, is given by component (D).

6. The edible microcarrier according to claim 1, wherein component (A) is crosslinked by salt cations selected from the group consisting of: Ca2+, Mg2+, Fe2+ and Fe3+ or a combination thereof.

7. The edible microcarrier according to claim 1, wherein said biopolymer in the microcarrier coating is a protein or a polypeptide or a polysaccharide or a combination thereof,
or wherein, if the coating protein, polypeptide or polysaccharide is positively charged at pH between 6-8, the microcarrier coating is applied directly on the microcarrier core, or, if the coating protein is negatively charged at pH between 6-8, a positively charged primer coating is applied first, wherein the primer coating is selected from a protein, a polypeptide or a polysaccharide or a combination thereof, more preferably from chitosan, chitin, polylysine or a combination thereof.

8. The edible microcarrier according to claim 1, wherein said crosslinking agent is selected from the group consisting of: transglutaminase, peroxidase, laccase, tyrosinase, lysyl oxidase, glutaraldehyde, genipin, citric acid, tannic acid or a combination thereof.

9. The edible microcarrier according to claim 1, wherein the edible microcarrier is dissolvable and/or degradable using a chelator or an enzyme, or a combination of an enzyme and a chelator,
and/or wherein said edible microcarrier has a spherical, cylindrical, fiber-shaped, ovoidal, or irregular shape
and/or wherein said microcarrier core has one dimension in the range of 0.05 mm and 2 mm.

10. A method of producing a coated edible microcarrier, including an edible microcarrier according to claim 1, for growing anchorage-dependent cells to prepare a cultured meat product, wherein the method comprises the steps of:
a) obtaining a precursor hydrogel solution of microcarrier core consisting of the following components:
(A) 0.03-10.0 wt % of at least one ionically cross-linkable polysaccharide;
(B) 0.05-5 wt % of a mechanical stability agent different from (A), or, if component (A) at the same time acts as a mechanical stability agent, 0.0-5 wt % of a mechanical stability agent different from (A);
(C) 0.0-10 wt % of additives different from (A) and (B);
(D) 0.0-50 wt % living cells;
(E) 75-99.92 wt % of water;
the sum of (A)-(E) making up 100 wt %;
b) forming droplets from said precursor hydrogel solution;
c) ionically crosslinking the droplets in a gelling solution through interactions with salt cations to form said microcarrier core.

11. The method according to claim 10, wherein in steps a) and b) the temperature of the solution is controlled in the range between 20° C. and 85° C.

12. The method according to claim 10, wherein the concentration of the salt cations in said gelling solution is comprised in the range between 20 mM and 1 M.

13. The method according to claim 10,
wherein before or after step (e) said coated edible microcarrier is immersed in a crosslinking bath,
and/or wherein, if not already adhered, cells are grown and/or adhered around the microcarrier to the microcarrier coating, and subsequently the edible microcarrier is dissolved and/or degraded using a chelator and/or an enzyme.

14. A method of using an edible microcarrier according to claim 1 for the preparation of a cultured meat product, including pork meat, beef meat, chicken meat, crustacea meat, fish meat.

15. A cultured meat product being based on or comprising an edible microcarrier according to claim 1, including pork meat, beef meat, chicken meat, crustacea meat, fish meat.

16. The edible microcarrier according to claim 1, wherein component (A) in said microcarrier core is a ionically crosslinked polyanionic polysaccharide, selected from the group consisting of: alginate, pectin, hyaluronan, gum arabic, xanthan gum and carboxymethylcellulose, carboxymethyl amylose, carboxymethyl chitosan, chondroitin sulfate, dermatan sulfate, heparin, heparin sulfate, and any of their salts, including sodium, potassium, magnesium, calcium, ammonium, or mixtures thereof and/or derivatives of any of these.

17. The edible microcarrier according to claim 1, wherein component (B) in said microcarrier core is selected from a thermo-responsive protein and/or a thermo-responsive polysaccharide, selected from the group consisting of: agar, agarose, carrageenan, xyloglucan, methylcellulose, gellan gum or mixtures thereof and/or derivatives of any of these.

18. The edible microcarrier according to claim 1, wherein component (C) in said microcarrier core is selected from the group consisting of: osmolarity regulating compounds; sugar alcohols; protein selected from gelatin, collagen, fibrinogen, fibrin, fibronectin, elastin, laminin, soy protein, zein protein, pea protein, canola protein, carob protein, cardosine A, wheat protein, albumin, casein protein, potato protein, guar protein; protein hydrolysates; mono- and oligosaccharides including natural mixes namely corn syrup, honey, maple syrup, glucose syrup, or a combination thereof, wherein the protein can be crosslinked using said crosslinking agent; or mixtures thereof and/or derivatives of any of these.

19. The edible microcarrier according to claim 18, wherein the component (C) in said microcarrier core is selected from the group consisting of an osmolarity regulating compound of a monosaccharide, a disaccharide, an oligosaccharide, or a combination thereof, or mixtures thereof and/or derivatives of any of these.

20. The edible microcarrier according to claim 1, wherein said microcarrier core comprises
component (A) in an amount of 0.7-5.5 wt %, or of 0.8-4-5 wt % or 0.8-3.5 wt %;
and/or component (B) in an amount of 0.15-1.5 wt %, or of 0.17-0.35 wt %;
and/or component (C) in an amount of 0.1-5 wt %, or of 1-4 wt %
and/or component (D) in an amount of 0.1-25 wt %, or of 1-10 wt %;
wherein in each case the complement, such that the sum of (A)-(E) is making up 100 wt %, is given by component (D).

21. The edible microcarrier according to claim 1, wherein component (A) is crosslinked by Ca2+.

22. The edible microcarrier according to claim 1, wherein said biopolymer in the microcarrier coating is a protein, selected from the group consisting of: gelatin, collagen, fibrinogen, fibrin, fibronectin, elastin, laminin, soy protein, zein protein, pea protein, canola protein, carob protein, cardosine A, wheat protein, albumin, casein protein, potato protein, guar protein, or a combination or derivative thereof,
and/or wherein, if the coating protein, polypeptide or polysaccharide is positively charged at pH between 6-8, the microcarrier coating is applied directly on the microcarrier core, or, if the coating protein is negatively charged at pH between 6-8, a positively charged primer coating is applied first, wherein the primer coating is selected from chitosan, chitin, polylysine or a combination thereof.

23. The edible microcarrier according to claim 1, wherein said crosslinking agent is transglutaminase.

24. The edible microcarrier according to claim 1, wherein at least one of the microcarrier core and the microcarrier coating, is dissolvable and/or degradable using a chelator or an enzyme or a combination of an enzyme and a chelator, wherein the chelator is EDTA or sodium citrate, and wherein dissolution is done with a chelator concentration of 1-100 mM within 1-90 min at a temperature between 4 and 40° C., and wherein the enzyme is an enzyme degrading the biopolymer, enzyme degrading the biopolymer including collagenase.

25. A method of producing an edible microcarrier according to claim 1, for growing anchorage-dependent cells to prepare a cultured meat product, wherein the method comprises the steps of:
a) obtaining a precursor hydrogel solution of microcarrier core, in the form of a solution consisting of the following components:
(A) 0.03-10.0 wt % of at least one ionically cross-linkable polysaccharide;
(B) 0.05-5 wt % of a mechanical stability agent different from (A), or, if component (A) at the same time acts as a mechanical stability agent, 0.0-5 wt % of a mechanical stability agent different from (A);
(C) 0.0-10 wt % of additives different from (A) and (B);
(D) 0.0-50 wt % living cells;
(E) 75-99.92 wt % of water;
the sum of (A)-(E) making up 100 wt %;
b) forming droplets from said precursor hydrogel solution, based on at least one of microfluidic droplet formation, electrostatic droplet formation, a vibration jet breakage device, a rotating jet breakage device, a co-axial airflow device, simple dropping, or water-in-oil emulsion;
c) ionically crosslinking the droplets in a gelling solution through interactions with salt cations to form said microcarrier core;
d) keeping the microcarrier core immersed in a solution, including in a solution containing 0.05%-3%, of a coating biopolymer to produce said coated edible microcarrier; and
e) optionally cooling down the edible microcarrier for thermal gelling of the microcarrier core
f) optionally immersing the microcarrier in a solution containing a crosslinking agent or adding a crosslinking agent to cross-link the coating biopolymer.

26. The method according to claim 10, wherein in steps a) and b) the temperature of the solution is controlled in the range between 55° C. to 80° C.

27. The method according to claim 10, wherein the concentration of the salt cations in said gelling solution is comprised in the range between 20 mM and 1 M, wherein the salt cations in said gelling solution are selected as Ca2+.

28. The method according to claim 10,
wherein before or after step (e) said coated edible microcarrier is immersed in a crosslinking bath, comprising crosslinking agent for the coating biopolymer in an amount of 1-20 or 5-10 mg/ml for a time span in the range of 1-300 minutes, or 50 minutes to 120 minutes,
and wherein the crosslinking agent is selected from transglutaminase, peroxidase, laccase, tyrosinase, lysyl oxidase, glutaraldehyde, genipin, citric acid, tannic acid or a combination thereof,
and/or wherein, if not already adhered, cells are grown and/or adhered around the microcarrier to the microcarrier coating, and subsequently at least one of the microcarrier core and the microcarrier coating is dissolved and/or degraded using a chelator or an enzyme, or a combination of an enzyme and a chelator, wherein the chelator can be EDTA or sodium citrate and wherein dissolution can be done with a chelator concentration of 1-100 mM within 1-90 min at a temperature between 4 and 40° C., and wherein the enzyme can be an enzyme degrading the biopolymer of the coating, including enzymes selected as collagenase, subsequently chelator and/or enzyme are removed from the solution and this solution is cross-linked, including by introducing into a cross-linking bath, to form beads with the cells encapsulated inside the previously dissolved microcarrier core material, optionally followed by cell differentiation to form the desired tissue.
